(19) United States
(12) Patent Application Publication
(10) Pub. No.: US 2009/0061029 A1
(43) Pub. Date: Mar. 5, 2009

(54) NIGHTTIME WATER

(76) Inventor: Kaira ROUDA, Columbus, OH (US)

Correspondence Address: STANDLEY LAW GROUP LLP, 6300 Riverside Drive, Dublin, OH, 43017, US

(21) Appl. No.: 12/200,282

(22) Filed: Aug. 28, 2008

Related U.S. Application Data
(60) Provisional application No. 60/968,379, filed on Aug. 28, 2007.

(51) Int. Cl. A61K 36/537; A61K 36/28; A61K 36/534

(52) U.S. Cl. 424/746; 424/764; 424/747

(57) ABSTRACT

Exemplary embodiments include a composition which can be added to drinking water and consumed to promote sleep. Embodiments may combine extracts of chamomile, lavender, poppy, valerian root, sage, ginko, and/or peppermint. The composition may be found on its own and added to drinking water by the end user. Alternatively, the composition may be found already in combination with drinking water, in a single serving or in multiple servings.

### CROSS-REFERENCE TO RELATED APPLICATIONS

This application is a non-provisional patent application and claims priority to co-pending application No. 60/968,379, filed on Aug. 28, 2007.

### TECHNICAL FIELD

The disclosed embodiments relate to a combination of materials which promote restful sleep.

### BACKGROUND AND SUMMARY

As our daily lives become more stressful and demanding, time becomes more and more precious. The never-ending search for more time in the day has fueled various consumer products aimed at speeding up daily tasks such as communicating with others, cooking, and cleaning. For those who still cannot find enough time in the day, there are products on the market to keep people awake and energized, such as energy drinks and caffeine pills. These types of products can provide people with more awake hours in the day, but it is done at the sacrifice of a good night's sleep. In this modern world where people suffer from stress and busy schedules, people are getting less sleep than necessary to function at their full potential. This sleep deficit can be detrimental to good health, as it not only affects a person's concentration and mood, but can also increase a person's chance of diseases such as diabetes. The recognition of the importance of sleep has fueled numerous types of sleeping pills. However, these pills can cause dependency, dizziness, and other unwanted side effects. All in all, a restful night's sleep has truly become the last frontier of luxury.

Along with sleep, people need a certain amount of water each day in order to function properly. In today's society many people ingest soda and coffee in huge quantities, and although they may have a good flavor, they are often diuretics and are one reason why many people suffer from dehydration. Another reason is availability. In many places the tap water is either unfit to drink or has a bad taste due to chlorine, minerals, or other contaminants. This is part of the reason why bottled water has become popular. Not only can it provide people with another source of drinking water that has a good taste, but it is also conveniently transportable.

As a result, it is an unmet advantage of the known art to provide bottled drinking water that enhances sleep.

One exemplary embodiment is a bottled water that can promote a restful night's sleep. Another exemplary embodiment is ingredients for promoting sleep which may be combined with water to produce a water mixture that can promote a restful night's sleep. Another exemplary embodiment is bottled water having ingredients with attractive flavors that may promote sleep. The mixture of water and ingredients may be available in large quantities (16 ounces or more) with multiple effective portions present within one large bottle. Alternatively, the mixture of water and ingredients may be available in a single portion.

Further embodiments package the ingredients for promoting restful sleep (and sometimes flavoring) separately from the water substrate. The ingredients may be available in a small container (three ounces or less) and may be combined with any form of drinking water (bottled or otherwise) by the consumer prior to consumption. These small packages of ingredients are particularly useful for travelers who, due to current FSA regulations, cannot bring a liquid container which is larger than 3 ounces onto an airplane. For many who travel, restful sleep is even more difficult, as jet lag and the general stress of traveling may deprive travelers of restful sleep. However, exemplary embodiments provide ingredients which can be easily portable during travel, and combined with any form of water at a later time, prior to consumption.

In addition to the novel features and advantages mentioned above, other features and advantages will be readily apparent from the following descriptions of the drawings and exemplary embodiments.

### DETAILED DESCRIPTION OF EXEMPLARY EMBODIMENTS

Considering the foregoing, an exemplary embodiment provides water containing natural ingredients that can promote a restful night's sleep. Another exemplary embodiment may be the ingredients themselves, which can be combined with drinking water by the consumer. Another exemplary embodiment is bottled water having herbal and fruit ingredients with attractive flavors that promote sleep. Examples of fruit and herbal ingredients may include, but are not limited to, lavender, valerian root, orange peel, passion flower, chamomile, blue poppy, cherry blossom, spearmint, cucumber, elderberry, raspberry leaf, and other similar or suitable ingredients.

Examples of herbal ingredients which may be used to promote sleep may include, but are not limited to, lavender, valerian root, spearmint, chamomile, linden flower, blue poppy, raspberry leaf, and cherry blossom. Additionally, any other herb that promotes restful sleep may also be used.

In some exemplary embodiments, herbal ingredients will be paired with fruit or vegetable ingredients in order to enhance the flavor of the water. Examples of fruits or vegetables that may be added to the water include, but are not limited to, orange peel, cucumber, and other similar or suitable ingredients derived or based on fruit or vegetables.

In certain embodiments, the water may contain pairs of herbal ingredients. Examples may include lavender paired with valerian root, chamomile with linden flower, elderberry with blue poppy, or raspberry leaf with cherry blossom. In other embodiments of the present invention an herbal ingredient may be paired with a fruit or vegetable flavor, such as orange peel and passion flower, or spearmint and cucumber. However, in other embodiments, different combinations of herbs, or different combinations of herbs and fruit and/or vegetable flavors may be used.

Chart 1 gives several embodiments which can provide an effective amount of the mixture so as to promote restful sleep and to provide a pleasant drinking flavor. The amounts of the ingredients shown may be combined with 8 ounces of drinking water. The ingredients may be found in combination with the water, or alternatively, the ingredients may be sold in the proper amounts separately, and combined with water by the ultimate user. It should be noted, that when the ingredients are packaged and available separate from the water substrate, this package may also include at least some water. For example, a package which contains a single serving of Combination 1 may not only contain 0.2 ml of chamomile, 0.2 of lavender, and 0.2 ml of poppy, but may also contain some water (for example 50 ml).

It should be noted that the exact amount of each ingredient may vary depending on the amount of water added and whether any additional flavors or substances are added. Chart 1 simply provides preferred effective combination ranges for a single serving for mixtures with 8 ounces of water. The combination of any of the ingredients, in a plurality of different amounts, would be within the scope of the exemplary embodiments. Some users may prefer a stronger flavor, where less than 8 ounces of water may be combined with the range of ingredients. Others may prefer a more diluted taste, where more than 8 ounces of water may be combined with the range of ingredients. It should also be noted that each of the ingredients is considered to be an essential oil.

CHART 1

| | | | |
|---|---|---|---|
| Combination 1 | .05-.2 ml | .05-.2 ml | .05-.2 ml poppy |
| | chamomile | lavender | extract |
| | extract | extract | |
| Combination 2 | .05-.2 ml ginko | .05-.2 ml sage | .025-.1 ml |
| | extract | extract | valerian |
| | | | root extract |
| Combination 3 | .25-1.0 ml poppy | .15-.6 ml sage | .025-.1 ml |
| | extract | extract | peppermint |
| | | | extract |
| Combination 4 | .05-.2 ml | | |
| | peppermint extract | | |
| Combination 5 | .1-.4 ml chamomile | | |
| | extract | | |
| Combination 6 | .1-.4 ml lavender | | |
| | extract | | |

The water used in the present invention may be any type of spring water, purified water, tap water, or other water that is desired. The ingredients can be found in a plurality of extract forms and combining the listed ingredients in any extract form is specifically contemplated in this application. Exemplary embodiments may utilize the various ingredients in the extract forms shown in Chart 2. Ingredients in the forms shown in Chart 2 are commercially available from Herb Pharm, Inc. of Williams, Oreg., which has a website of www.herb-pharm.com.

CHART 2
lavender extract = Liquid herbal extract. Lavandula angustifolia - provence-grown
flowers. Dry flower/menstruum ratio = 1:5. Contains certified organic grain alcohol (78-84%),
distilled water & Lavender extractives.
chamomile extract = Liquid herbal extract. Matricaria recutita fresh flower. Fresh herb/
menstruum ratio = 1:1.25. Contains certified organic grain alcohol (50-60%), distilled
water and Chamomile extractives.
peppermint extract = Peppermint Spirits - Liquid herbal extract; essential oil & whole
leaf extract - Contains certified organic grain alcohol (81-87%), Peppermint essential
oil & peppermint extractives.
poppy extract = liquid herbal extract/herbal dietary supplement - Eschscholzia
Californica - Fresh Root, Leaf & Flower - Contains certified organic grain alcohol (54-64%),
distilled water & California Poppy Extractives. Fresh herb/menstruum ratio -
1:1.75
valerian root extract = liquid herbal extract. Valeriana officinalis Fresh Rhizome &
Root. Fresh herb/menstruum ratio = 1:1.25. Contains certified organic grain alcohol
(49-59&) & Valerian extractives
sage extract = liquid herbal extract. Salvia officinalis. Dry herb/menstruum ratio: 1:5.
Contains certified organic grain alcohol (70-75%), distilled water and sage extractives.
ginko extract = liquid herbal extract. Ginkgo Biloba autumn harvested leaf. Dry herb/
menstruum ratio = 1:5. Contains certified organic grain alcohol (57-67%) distilled water,
vegetable glycerine & Ginkgo extractives.

Having described exemplary embodiments, those skilled in the art will realize that many variations and modifications may be made to affect the exemplary embodiments and still be within the scope of the claimed invention. Thus, many of the elements indicated above may be altered or replaced by different elements which will provide the same result and fall within the spirit of the exemplary embodiments. It is the intention, therefore, to limit the invention only as indicated by the scope of the claims.

I claim:

1. A composition for oral ingestion in a human, comprising:
an effective amount of at least one essential oil to promote sleep.

2. The composition of claim 1, wherein:
the at least one essential oil comprises chamomile, in an extract.

3. The composition of claim 2, further comprising:
potable water, in the range of between 200 ml and 1200 ml.

4. The composition of claim 2, wherein:
the at least one essential oil further comprises lavender and poppy, both in an extract.

5. The composition of claim 1, wherein:
the at least one essential oil comprises peppermint, in an extract.

6. The composition of claim 5, further comprising:
potable water, in the range of between 200 ml and 1200 ml.

7. The composition of claim 5 wherein the at least one essential oil further comprises sage and poppy, both in an extract.

8. The composition of claim 7, further comprising:
potable water, in the range of between 200 ml and 1200 ml.

9. The composition of claim 1, wherein:
the at least one essential oil comprises lavender, in an extract.

10. The composition of claim 9, further comprising:
potable water, in the range of between 200 ml and 1200 ml.

11. The composition of claim 1, wherein:
the at least one essential oil comprises ginko, sage, and valerian root, all in extract.

12. The composition of claim 11, further comprising:
potable water, in the range of between 200 ml and 600 ml.

13. A composition for oral ingestion to promote sleep in a human, comprising:
potable water and chamomile extract, wherein said chamomile extract provides no more than 0.5% of the composition by volume.

14. The composition of claim 13 wherein said chamomile extract is a hydroalcoholic extract.

15. The composition of claim 13 further comprising: lavender and poppy extracts, wherein:
said lavender extract provides no more than 0.5% of the composition by volume; and
said poppy extract provides no more than 0.5% of the composition by volume.

16. The composition of claim 15 wherein said chamomile, lavender, and poppy extracts are hydroalcoholic extracts.

17. A composition for oral ingestion to promote sleep in a human, comprising:
potable water and chamomile extract, wherein said peppermint extract provides no more than 0.5% of the composition by volume.

18. The composition of claim 17 wherein said peppermint extract is a hydroalcoholic extract.

19. The composition of claim 17 further comprising: poppy and sage extracts, wherein:
said sage extract provides no more than 0.5% of the composition by volume; and
said poppy extract provides no more than 0.8% of the composition by volume.

20. The composition of claim 19 wherein said peppermint, poppy, and sage extracts are hydroalcoholic extracts.
